(19) United States
(12) Patent Application Publication
(10) Pub. No.: US 2007/0167862 A1
(43) Pub. Date: Jul. 19, 2007

(54) Cardiac output measurement devices and methods

(76) Inventor: George A. Lopez, Laguna Beach, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/417,579

(22) Filed: May 3, 2006

Related U.S. Application Data
(60) Provisional application No. 60/740,543, filed on Nov. 29, 2005.

(51) Int. Cl. A61B 5/00

(52) U.S. Cl. 600/549

(57) ABSTRACT

A device for measurement of cardiac output including an elongate body and a plurality of electrical components, including at least an energy producing element, such as a heating coil, and a temperature sensing element, such as a thermistor. The elongate body includes a plurality of electrical lead wires configured to transmit electric current to the electrical components and at least one insulation layer configured to electrically insulate the plurality of lead wires from one another. Preferably, a cross-sectional size of the elongate body is generally equal to a combination of the cross-sectional size of the plurality of lead wires and a cross-sectional size of the at least one insulation layer. In a preferred method of use, the device is introduced into the radial artery of a patient.

### CROSS-REFERENCE TO RELATED APPLICATION

This application claims the benefit of U.S. Provisional Application No. 60/740,543, filed Nov. 29, 2005, which is incorporated by reference in its entirety.

### BACKGROUND OF THE INVENTIONS

1. Field of the Inventions

The present inventions relate to devices and methods for measurement of the cardiovascular system of a patient. More specifically, the present inventions relate to devices and methods for measuring cardiac output and, in some arrangements, measuring other blood parameters.

2. Description of the Related Art

The monitoring of cardiac output is a common diagnostic technique used to evaluate the heart function and fitness of a patient. Cardiac output is sometimes defined as the volume of blood pumped by the heart over a period of time and is typically expressed in units of liters per minute (L/min.). Multiple techniques exist to measure or estimate cardiac output. However, the existing methods suffer from one or more disadvantages, including lack of precision, discontinuous or interrupted data collection, high risk of infection, and significant discomfort and inconvenience to the patient.

One method of measuring cardiac output is known as thermodilution. This method involves producing a temperature change at one point in a blood vessel and measuring the temperature of the blood at a second point in the vessel. The measured change in temperature between the first point and the second point provides an indication of the blood flow volume through the vessel. In practice, thermodilution devices and methods have generally been used within a catheter lodged in a patient's blood vessel. Such catheters may include a heating element and a temperature measurement element. A thermodilution catheter is sometimes advanced through the vessel so that it resides at least partially in a heart chamber. The catheter is generally configured to allow blood from the vessel to flow inside of the catheter (typically by way of openings in the catheter wall).

The heating element produces a temperature change in the blood within the catheter in the vessel. The temperature change is measured by the temperature sensing element, usually located in the catheter, at a point in the blood vessel downstream from the heat producing element. As used herein, the term “upstream” refers to the direction from which blood flow originates within a blood vessel, and “downstream” refers to the direction where blood flow is going within a blood vessel. The temperature change and the quantity of heat introduced to the blood are utilized to determine the blood flow rate within the vessel through a mathematical relationship.

Although certain conventional thermodilution methods can be relatively accurate in some applications and circumstances, such methods have many shortcomings. For example, if thermodilution catheters remain within the patient for an extended period of time, the risk of infection becomes significant. It has been estimated that the cost of treating infections caused by thermodilution catheters can be many times the combined cost of the catheter and the implantation procedure. Furthermore, while the thermodilution catheters are in place, the mobility of the patients may be significantly restricted. In addition, the presence of a catheter for an extended period of time is likely to be uncomfortable for the patient because the diameter of the catheter is typically relatively large in comparison with the diameter of a blood vessel. The large size of the catheter can also cause trauma, damage, and other interference within the vessel by contacting internal issues and impeding blood flow.

Another method for determining cardiac output involves monitoring a patient's “whole body oxygen consumption.” In this method, a first probe is generally placed within an artery of the patient and a second probe is placed within a vein of the patient. The oxygen content of the arterial blood is compared with the oxygen content of the venous blood in order to estimate the body's overall oxygen consumption. The whole body oxygen consumption estimate is then used to estimate the cardiac output. This method has many disadvantages as well. The method typically depends upon several assumptions about the patient's overall body characteristics and also involves averaging several blood parameters. The body's oxygen consumption is not a fixed value, but tends to fluctuate, even if cardiac output remains constant. Accordingly, the use of whole body oxygen consumption to estimate cardiac output may lead to undesirable errors and delays in the reporting of cardiac output events. Although the individual arterial and venous probes used in this method may be smaller than in the typical thermodilution method, multiple access points are generally required in order to collect data from both an artery and a vein.

### SUMMARY OF THE INVENTIONS

Some embodiments of the present inventions include a blood measuring component with an energy producing element, a temperature sensing element, elongate electrical leads connected to each of these elements, and one or more coatings (such as electrical or thermal insulator coatings) surrounding at least a portion of the elements and/or leads. A catheter body is generally not required to support the electrical components or lead wires. As a result, the cross-sectional dimensions of the portion of the device inside of the patient can be greatly reduced in comparison to typical thermodilution catheters. In addition, smaller blood vessels can be utilized, such as the radial artery, for example. Furthermore, the risk of infection can be greatly reduced because the portion of the blood measuring component that passes through the skin of the patient is generally much smaller than in a typical thermodilution method. In such an arrangement, the device can be implanted for relatively long periods of time with minimized risk of infection and discomfort to the patient. Moreover, the patient's pain upon insertion and the discomfort of prolonged usage can be significantly diminished.

In some embodiments of a cardiac output measuring device, the blood measurement component or probe includes an energy producing element and a first pair of lead wires configured to transmit electric current through the energy producing element. The device also can include a temperature sensing element and a second pair of lead wires configured to transmit electric current through the temperature sensing element. At least one coating can be configured to electrically insulate each wire of the first and second pairs of lead wires from one another. The coating(s) can provide electrical insulation of the lead wires, energy producing element, and/or temperature sensing element. The coating(s) can also impart a desired degree of stiffness to the blood measurement component to achieve a particular positioning or orientation of the component within the blood vessel. The coatings(s) can also include one or more substances that produce or enhance antimicrobial or anticoagulant effects. The first and second pairs of lead wires can be secured in an elongate bundle.

In some embodiments, a device for measuring cardiac output includes a probe connected to a controller configured to calculate cardiac output utilizing information regarding a quantity of energy introduced to blood within the vasculature by the energy producing element and a change in a temperature of the blood detected by the temperature sensing element. In some embodiments, a first transceiver is electrically connected to the energy producing element and the temperature sensing element. A second transceiver is electrically connected to the controller. The first transceiver and the second transceiver communicate with one another over a wireless connection to transmit control signals and data signals between the controller and the probe.

A method of determining a cardiac output of a patient includes accessing an artery of the patient such as the radial artery. A probe is positioned within the radial artery and is used to introduce a quantity of heat to the blood within the radial artery. The probe is also used to measure a temperature change within the radial artery. The cardiac output is calculated by the controller based on the quantity of heat introduced and the temperature change.

### BRIEF DESCRIPTION OF THE DRAWINGS

These and other features, aspects, and advantages of the present inventions are described below with reference to drawings of several embodiments, which are intended to illustrate, but not to limit, the present inventions.

FIG. 1 illustrates a cardiac output measurement device introduced into a blood vessel of a patient for monitoring cardiac output.

FIG. 2 illustrates a portion of the cardiac output measurement device positioned within the radial artery of the patient.

FIG. 3A is a schematic illustration of a portion of a cardiac output measurement device within the radial artery of the patient.

FIG. 3B is an enlarged schematic illustration of a portion of the cardiac output measurement device of FIG. 3A, taken along view line 3B.

FIG. 4 is an enlarged, partial view of a distal end portion of a cardiac output measurement device in which the lead wires are joined by a coating.

FIG. 5 is a cross-sectional view of a configuration for lead wires in a cardiac output measurement device, taken along view line 5-5 in an alternative arrangement of FIG. 4.

FIG. 6 is a schematic illustration of another embodiment of a cardiac output measurement device. The cardiac output measurement device includes additional components, such as sensors, to permit the monitoring of other blood parameters.

FIG. 7 is a schematic illustration of another embodiment of a cardiac output measurement device. The cardiac output measurement device is configured for wireless communication with a controller to provide for increased freedom of movement for the patient.

### DETAILED DESCRIPTION OF THE PREFERRED EMBODIMENTS

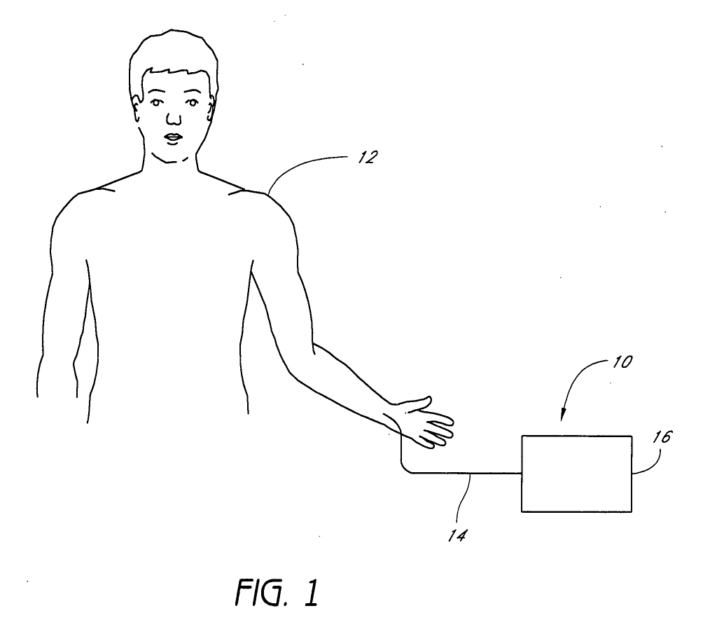

FIGS. 1-5 illustrate embodiments of cardiac output measurement devices. With reference to FIG. 1, the cardiac output measurement device 10 is configured for use in monitoring the cardiac output of a patient 12. The device 10 includes a probe 14 connected to a controller 16. The probe 14 is introduced within the vasculature of the patient 12 and is configured to detect certain parameters of the blood within the vasculature of the patient 12. Data collected by the probe 14 is communicated to the controller 16, which utilizes the data to calculate the cardiac output of the patient 12 and/or other desired physiological parameters of the patient 12. Furthermore, the controller 16 can be configured to send control signals to the probe 14, as described in further detail below.

The probe 14 is configured to produce a temperature change to the blood within the vasculature of the patient 12, which generally involves adding heat energy to the blood. The controller 16 communicates a control signal to the probe 14, such as an electrical current, to activate the probe 14 to introduce a quantity of heat in the blood. The probe 14 also detects the temperature in a localized area in the blood and communicates this data to the controller 16. This measured temperature can be used to calculate the difference between the known temperature in the region in the blood near the heat-producing element and the region in the blood near the temperature-sensing element. The controller 16 uses the data supplied by the probe 14 to calculate the cardiac output of the patient 12 using a mathematical relationship between cardiac output to the addition of energy to the blood and the resulting temperature change.

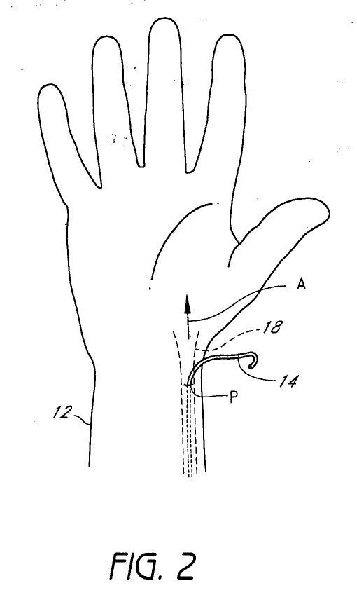

FIG. 2 illustrates one method of placing the probe 14 within the vasculature of the patient. In the illustrated arrangement, the probe 14 is introduced into the radial artery 18 of the patient 12 in the region of one of the patient's wrists. The radial artery 18 is near the surface of the skin and thus conveniently accessible for placement of the probe 14. Many other suitable blood vessels can also be used, including arteries and veins, as well as heart chambers, if desired. For example, in some arrangements, the probe 14 may be configured for use in the brachial artery or femoral artery.

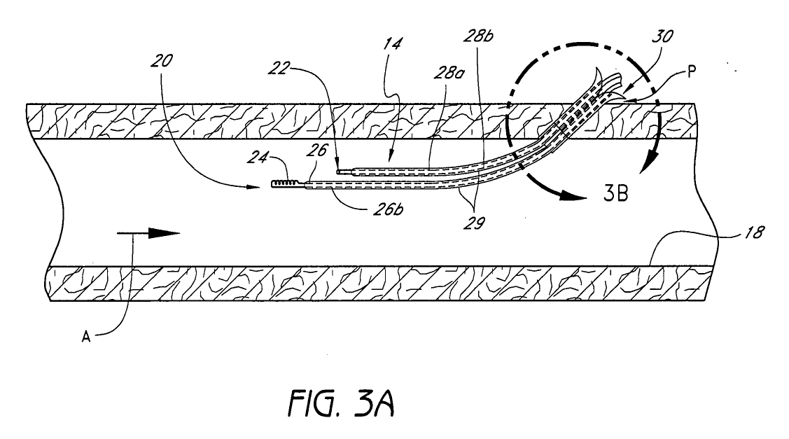

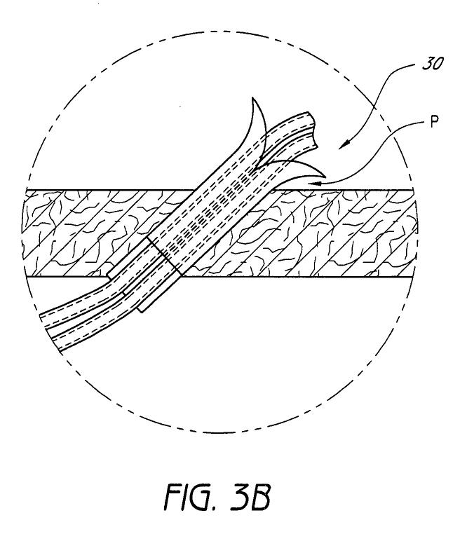

The probe 14 can be introduced into the radial artery 18 through the skin of the patient 12 at an access point P. In the illustrated arrangement, the probe 14 is advanced within the artery 18 after insertion in a direction toward the heart of the patient 12. In other words, the probe 14 is advanced upstream within the artery 18 in a direction opposite of the direction of blood flow within the artery 18, which is indicated by the arrow A in FIGS. 2 and 3. In other embodiments, the probe 14 can be configured to be advanced within the blood vessel in the direction of blood flow. In such embodiments, the relative positions of energy producing elements and temperature sensing elements on the probe 14 can be modified, as explained in greater detail below.

FIG. 3A illustrates an end of the probe 14 within the radial artery 18 of the patient 12. As described above, the probe 14 preferably includes an energy producing element 20 and an energy sensing element 22. The energy producing element 20 is configured to introduce a certain amount of energy into the blood within the artery 18. In some embodiments, the energy producing element 20 is configured to introduce heat into the artery 18 and includes a heating coil 24, and the energy sensing element 22 is configured to measure temperature. The probe 14 can be adapted to introduce other suitable types of energy into the blood. Furthermore, other suitable types of heat producing devices may be used.

In the illustrated arrangement, a pair of lead wires, 26A and 26B respectively, are connected to opposing ends of the heating coil 24. The lead wires 26A, 26B can extend through the artery 18 and outside of the patient 12 at the access point P. The lead wires 26A, 26B are connected to the controller 16 by any suitable connection to permit electrical communication between the heating coil 24 and the controller 16. In some embodiments, the lead wires 26A, 26B and the heating coil 24 can be constructed of a single wire, which may be a single filament wire or a multifilament wire. The lead wires 26A, 26B and heating coil 24 can be constructed of the same or different materials. Any suitable material or combination of materials known to those of skill in the art can be used in the fabrication of the heating coil 24 and lead wires 26A and 26B, such as nickel or platinum, for example.

In some embodiments, the temperature sensing element 22 is a thermistor, or a thermally-sensitive resistor. The thermistor may be a positive or negative thermistor. The resistance of a positive thermistor increases with an increase in temperature and the resistance of a negative thermistor decreases with an increase in temperature. A thermistor is desirable for its simplicity. Other suitable temperature sensing devices can also be used.

The temperature sensing element 22 includes a pair of lead wires 28A and 28B, which extend from the thermistor 22, through the artery 18, and exit the patient at the access point P. The lead wires 28A and 28B are connected to the controller 16 by any suitable connection to permit electrical communication between the thermistor 22 and the controller 16. The lead wires 28A and 28B may be comprised of any suitable material, or combination of materials, for transmitting a signal from the thermistor 22 to the controller 16, such as nickel or platinum, for example. The lead wires, 26A, 26B, 28A, 28B, either individually or in pairs, can be coated with electrically and/or thermally insulating material 29.

As illustrated in FIG. 3A, the energy producing element 20 is upstream in the blood flow from the temperature sensing element 22. When the end of the probe 14 is oriented upstream from the access point P (as illustrated in FIG. 3A), the energy producing element 20 is located further from the access point P than the temperature sensing element 22. When the end of the probe 14 is oriented downstream from the access point P (not shown), the energy producing element 20 is located closer to the access point P than the temperature sensing element 22.

The probe 14 can include other components (not shown in FIG. 3A), such as additional heating coils 24 (or other energy producing devices) and/or additional thermistors 22 (or other temperature sensing devices). For example, an additional thermistor can be positioned nearer the heating coil 24 to detect a temperature of the blood near the heating coil 24. Such data can be used, for example, to estimate, or to verify, the quantity of heat introduced to the blood by the heating coil 24.

Furthermore, in some arrangements, an additional heating coil may be provided as a “dummy load,” which would preferably be positioned outside of the blood vessel 18. The dummy load can be connected to at least one of the lead wires 26A or 26B and can be activated inversely of the heating coil 24; when the heating coil 24 is on, the dummy load would be off and vice-versa. As a result, the electrical current through the lead wires 26A and 26B would be constant to reduce the opportunity for error in the temperature measurement caused by heat from the lead wires 26A, 26B affecting the thermistor 22.

As illustrated in FIGS. 3A and 3B, an introducer 30 may be used to provide access to the radial artery 18. The introducer 30 can be initially introduced through the wall of the artery 18 with the assistance of a needle positioned partially within the interior of the introducer 30 (not shown). The needle has a sharp tip that extends beyond an end of the introducer 30 for piercing the wall of the artery 18. The needle preferably defines an internal passage, which permits the probe 14 to be passed therethrough and into the artery 18. The needle can be subsequently withdrawn, and the probe 14 and introducer 30 remain. In the illustrated embodiment, the introducer 30 is slightly wider than the combined widths of the coated lead wires 26A, 26B, 28A, 28B. In other embodiments, the introducer 30, or at least the portion thereof positioned outside of the body during use, can be substantially larger, e.g., at least about 1.5 times, at least about 2 times, at least about 2.5 times, at least about 3 times, at least about 3.5 times, at least about 4 times, at least about 4.5 times, or at least about 5 times larger, than the combined widths of the coated lead wires 26A, 26B, 28A, 28B, to facilitate manually inserting and manipulating the introducer 30 without the need to increase the size of the lead wires. The introducer 30 can comprise a multiple section, or peel-away, needle, which is configured for separation into two or more halves to permit the introducer 30 to be removed from the probe 14 once the probe 14 has been inserted into the artery 18. By separating into two or more halves, the introducer 30 does not have to be sized to pass over any connectors at the end of the probe 14. However, in alternative arrangements, other suitable methods of introduction of the probe 14 to the blood vessel 18 may be used.

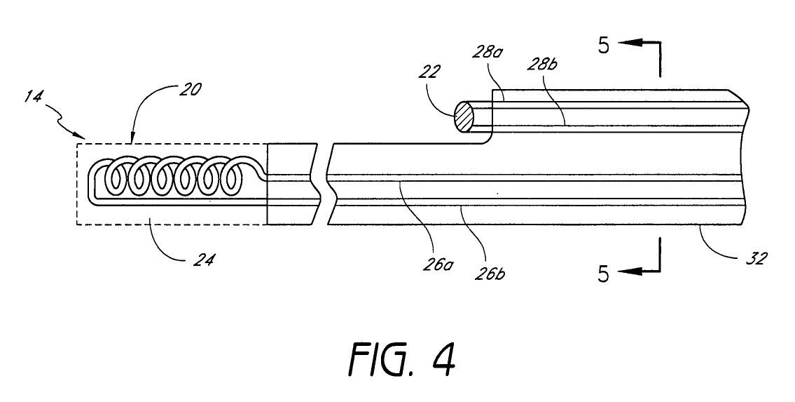

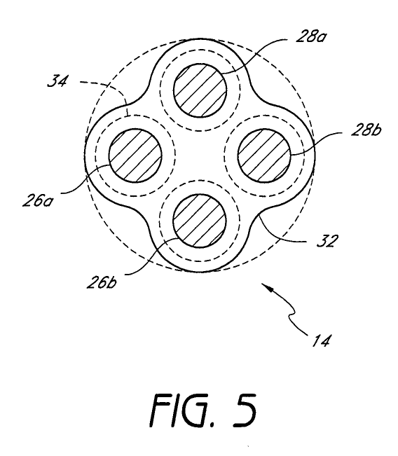

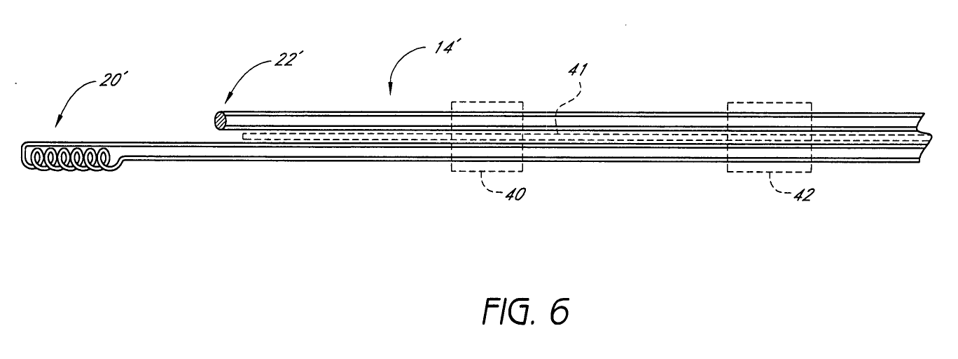

With reference to FIGS. 4 and 6, at least a portion, and preferably a substantial portion of the length of the lead wires 26A, 26B and 28A, 28B are covered and/or surrounded by a coating 32. The coating 32 can insulate the lead wires 26A, 26B and 28A, 28B from one another and from the patient 12. The coating 32 can provide electrical insulation and, in some arrangements, can provide at least some amount of thermal insulation. Furthermore, the coating 32 can provide some structural support for the lead wires 26A, 26B and 28A, 28B to help keep the ends of the probe 14 located near the central portion of the blood flow and to maintain a desired spacing and/or orientation between the heating element 24 and the temperature sensing element 22. The lead wires 26A, 26B and 28A, 28B can be spaced from each other as shown in FIGS. 4 or 5, or the lead wires 26A, 26B and 28A, 28B can be positioned adjacent to each other, or in other suitable spacing arrangements. The coating 32 can be constructed from any suitable material selected to provide the desired properties and/or provide a desired degree of stiffness or column strength for the probe 14. Examples of materials that may be suitable in some applications are various polymers, silicone, epoxy, and/or other adhesives. In addition, the coating 32 material may also include materials with therapeutic properties, such as agents with specialized functions. In one example, the coating 32 may include sodium nitro-prusside, or other materials designed to avoid clotting, reduce infection risks, and/or encourage regrowth of damaged tissue.

In some embodiments, the coating 32 terminates prior to the thermistor 22, such that the thermistor 22 is external of the coating 32 and, accordingly, is disposed directly within the blood of the artery 18 to sense changes in temperature of the blood. The coating 32 may terminate prior to the heating coil 24 such that the heating coil 24 is directly in contact with blood. However, if desired, the coating 32 may encapsulate the heating coil 24 and/or the thermistor as illustrated by the dashed lines of FIG. 4. In such an arrangement, the coating 32 (or at least the portion of the coating 32 covering the heating coil 24) can permit heat to be passed from the heating coil 24 through the coating 32 and to the blood within the artery 18. Such an arrangement may inhibit clotting of the blood on the heating coil 24 and/or disruption of the blood flow, for example. The portion of the coating 32 covering the heating coil 24 can be constructed of a different material than the remainder of the coating 32. Furthermore, in certain arrangements, the coating 32 may include multiple materials or multiple layers in accordance with the desired properties of the probe 14.

With reference to FIG. 5, the lead wires 26A, 26B and 28A, 28B are preferably bundled so as to be relatively compact in a plane transverse to the longitudinal axis of the probe 14. As noted above, the coating 32 preferably surrounds all of the lead wires 26A, 26B and 28A, 28B and separates them from one another. However, preferably there is no cannula or lumen defined within the probe 14. Accordingly, the radial dimension of the probe 14 is determined primarily by the diameter of the lead wires 26A, 26B and 28A, 28B (and other components, including additional lead wires or other elements) and the desired thickness of the coating 32 (including multiple coating layers). As a result, the cross-sectional width of the probe 14 preferably is less than the cross-sectional size of typical thermodilution catheters and, more preferably, only a fraction of the cross-sectional size of such catheters. For example, in some embodiments, the individual or combined widths of one or more of the lead wires 26A, 26B, 28A, 28B (with or without coatings) can be similar in size to a human hair, e.g., less than about 20μ, less than about 50μ, less than about 100μ, less than about 200μ, or somewhat larger than a human hair, e.g., less than about 400μ, less than about 600μ, or less than about 800μ. In the illustrated embodiment, the cross-sectional width of the combined, coated lead wires 26A, 26B, 28A, 28B is substantially smaller than the diameter of the radial artery (e.g., about one-tenth the size). Thus, if the radial artery diameter is about 1.5 mm, then the cross-sectional width of the combined, coated lead wires 26A, 26B, 28A, 28B is about 150μ. In other embodiments, the proportion of the cross-sectional widths of one or more of the lead wires 26A, 26B, 28A, 28B (with or without coatings) to the radial artery diameter can be smaller, e.g., less than about 1/10, or larger, e.g., between about 1/10 and about ¼, less than about ¼, between about ¼ and about ½, or less than about ½. As with all quantities provided herein, other sizes and proportions within and outside of these ranges can also be used. Preferably, the volume of blood between the vessel wall and the heating element 24 and/or the temperature sensing element 22, and any associated coatings, does not include any other structures associated with the probe 14 to impede or otherwise interfere with blood flow. Accordingly, in many cases, the probe 14 can be implanted with less discomfort to the patient, reduced risk of infection, less blood flow turbulence, reduced risk of blockage or clotting in blood flow, and/or reduced risk of trauma or interference with the vessel wall and other body structures and/or tissues (which can be especially desirable if the probe 14 is advanced to a position near or inside of the heart), in comparison to conventional thermodilution catheters. These advantages, either alone or in combination, can permit the probe 14 to remain within the patient for a much longer period of time, and hence cardiac output may be continuously monitored with diminished discomfort and without interruption for a much longer period of time.

The coating of the probe 14 may take on a number of suitable arrangements. As illustrated in FIG. 5, each of the lead wires 26A, 26B and 28A, 28B may be coated individually, as indicated by a dashed line in FIG. 5 and labeled with the reference number 34. The individually coated lead wires 26A, 26B, and 28A, 28B may then be secured to one another, for example by the coating 32. In such an arrangement, the coating 32 may extend the entire length of the probe or may be provided intermittently to secure the coated lead wires 26A, 26B and 28A, 28B together.

As illustrated in FIG. 5, the probe 14 may take on a variety of cross-sectional shapes. For example, the shape may be determined by the general shape of the bundled lead wires 26A, 26B and 28A, 28B and, thus, may vary with the number of lead wires present. Such an arrangement of the coating 32 is illustrated in solid line in FIG. 5. As alternatively illustrated by a dash line, the coating 32 may be configured to provide the probe 14 with a desired cross-sectional shape, such as the generally circular shape illustrated, regardless of the general shape of the bundled lead wires 26A, 26B and 28A, 28B. The coating 32 (or coating 34) may be applied to the lead wires 26A, 26B and 28A, 28B by any suitable method. For example, the coatings 32 or 34 may be applied by dipping, spraying, deposition, extrusion, shrink-fit or any other suitable process.

In use, preferably the device 10 is utilized to monitor the cardiac output of the patient 12. The introduction needle 30 is used to access the radial artery 18 of the patient 12. The probe 14 is introduced through the introducer needle 30 into the radial artery 18. Once the probe 14 is positioned within the radial artery 18, the introducer needle 30 may be withdrawn and, desirably, separated into two halves or otherwise removed from the probe 14.

The probe 14 may be connected to the controller 16, which is configured to provide operating signals to the probe 14 and receive data signals from the probe 14. The controller 16 provides a signal to operate the heat producing element 20 such that a desired quantity of heat is introduced to the blood within the radial artery 18. The temperature sensing element 22 then senses the temperature of the blood within the radial artery 18 at a point downstream from the heat producing element 20. The temperature sensing element 22 sends a signal corresponding to the temperature to the controller 16. Using the known heat imparted to the blood and the resulting drop in temperature at the point downstream from the heat producing element 20, the controller 16 uses an appropriate algorithm to determine the cardiac output, or volume flow of blood per unit of time.

Due to the relatively small cross-sectional dimension of the probe 14, in some embodiments, the probe 14 may be left in place within the radial artery 18 for an extended period of time to permit continuous monitoring of the cardiac output of the patient 12 without significant patient discomfort or risk of infection. This represents a significant improvement over the thermodilution catheters of the prior art, which tend to have significant discomfort and costs associated with related infection rates and limited mobility of the patient.

FIG. 6 illustrates another embodiment of a probe and is referred to by the reference numeral 14′. The probe 14′ is similar in some respects to the probe 14 and, therefore, like reference numerals are used to denote like components, with the exception that a prime (′) is added. The probe 14′ also includes an energy producing element 20′ and a temperature sensing element 22′. The probe 14′ also includes additional components or sensors that may be used to monitor other physiological parameters of the patient 12. One such sensor can be a blood gas sensor 40, which can be configured to monitor one or more of common blood gas values, such as oxygen saturation, partial oxygen, partial carbon dioxide and bicarbonate. Although illustrated as a single sensor, the illustrated sensor 40 may be comprised of multiple sensors. The controller 16′ (not shown) may be configured to receive data from the blood gas sensor 40 and, preferably, compute both directly measured values and those values that are calculated from the directly measured values.

In one arrangement, the device 10′ may include one or more fiber optic probes 41 (one shown) to measure additional blood parameters, such as venous partial 0₂, for example. In such an arrangement, the fiber optic probe 41 may also provide some degree of stiffness, or column strength, to the probe 14′. Accordingly, the coating 32′ may be provided largely for an insulation function such that the thickness may be minimized. Furthermore, additional sensors 42 may also be provided to detect other physiological variables of the blood, such as the blood PH level for example.

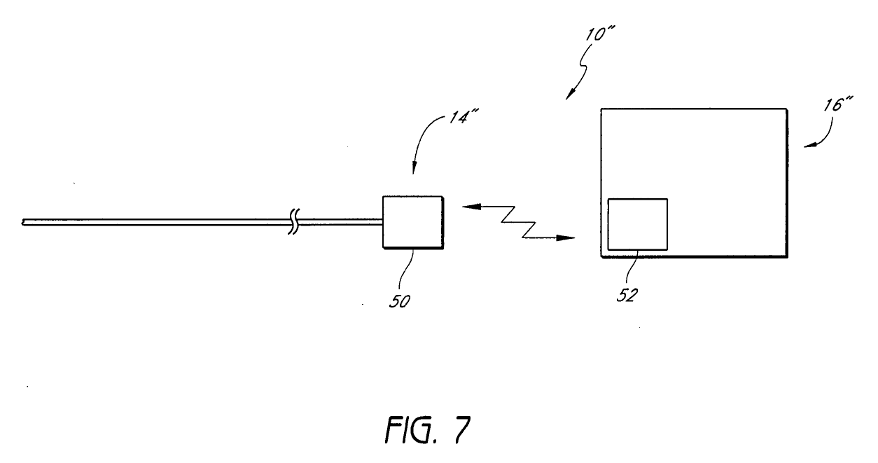

FIG. 7 illustrates another embodiment of a probe and is indicated by the reference numeral 14″. The device 10″ of FIG. 7 is similar in some respects to the device 10 of FIGS. 1-5 and, accordingly, like reference numerals indicate like components, with the exception that a double prime (″) is added. In the device 10 , a transceiver 50 is electrically connected to the probe 14″ and is configured to communicate with a transceiver 52 of the controller 16″. Preferably, the transceiver 50 and transceiver 52 communicate over a wireless connection, which may follow a suitable communication protocol, such as a Bluetooth communication protocol, for example. However, other suitable types of wireless communication may also be used.

With such a system, control and data signals may be communicated between the probe 14″ and the controller 16″, through the transceivers 50, 52, such that the device 10″ may operate substantially as described above. Advantageously, the device 10″ generally affords the patient 12 more mobility relative to the controller 16″ for increased comfort and convenience. As will be appreciated, either of the transceivers 50, 52 may be replaced by a transmitter or receiver, as appropriate, if only one-way communication is necessary or desired. Furthermore, in some arrangements, operational functions of the controller 16″ and probe 14″ may be otherwise separated, or performed by additional system components, as may be desirable.

Although this invention has been disclosed in the context of certain preferred embodiments and examples, it will be understood by those skilled in the art that the present invention extends beyond the specifically disclosed embodiments to other alternative embodiments and/or uses of the invention and obvious modification and equivalents thereof. In particular, while the present cardiac output monitoring device and method have been described in the context of particularly preferred embodiments, the skilled artisan will appreciate, in view of the present disclosure, that certain advantages, features and aspects of the system may be realized in a variety of other applications, many of which have been noted above. Additionally, it is contemplated that various aspects and features of the invention described can be practiced separately, combined together, or substituted for one another, and that a variety of combinations and subcombinations of the features and aspects can be made and still fall within the scope of the invention. Thus, it is intended that the scope of the present invention herein disclosed should not be limited by the particular disclosed embodiments described above, but should be determined only by a fair reading of the claims.

1. A device for measurement of cardiac output, comprising:
a probe configured to be positioned within vasculature of a patient, said probe comprising an energy producing element and a temperature sensing element;
a controller configured to calculate cardiac output utilizing information regarding a quantity of energy introduced to blood within the vasculature by the energy producing element and a change in a temperature of the blood detected by the temperature sensing element;
a first transceiver secured to said probe and electrically connected to said energy producing element and said temperature sensing element;
a second transceiver electrically connected to the controller, wherein said first transceiver and said second transceiver communicate with one another over a wireless connection such that control signals and data signals may be transmitted between said controller and said probe.

2. The device of claim 1, wherein said probe comprises an elongate body and a plurality of electrical components, which includes said energy producing element and said temperature sensing element, said elongate body comprising a plurality of electrical lead wires connected to said plurality of electrical components and at least one insulation layer configured to electrically insulate said plurality of lead wires, wherein a cross-sectional size of said elongate body is generally equal to a combination of the cross-sectional size of said plurality of lead wires and a cross-sectional size of said at least one insulation layer.

3. The device of claim 1, wherein said energy producing element comprises a heating element.

4. The device of claim 1, wherein said temperature sensing element comprises a thermistor.

5. The device of claim 1, wherein said energy producing element is positioned distally of said temperature sensing element on said device.
